(19) United States
(12) Patent Application Publication
(10) Pub. No.: US 2024/0418622 A1
(43) Pub. Date: Dec. 19, 2024

(54) pH ESTIMATION DEVICE AND pH ESTIMATION METHOD FOR SILICA-SUPERSATURATED FLUID

(71) Applicant: FUJI ELECTRIC CO., LTD., Kawasaki-shi (JP)

(72) Inventors: Taichiro KATO, Tokyo (JP); Shinya UI, Tokyo (JP); Azusa OZAWA, Tokyo (JP); Tianlong JIANG, Tokyo (JP); Fumi UI, Tokyo (JP)

(73) Assignee: FUJI ELECTRIC CO., LTD., Kawasaki-shi (JP)

(21) Appl. No.: 18/817,780

(22) Filed: Aug. 28, 2024

Related U.S. Application Data
(63) Continuation of application No. PCT/JP2023/031952, filed on Aug. 31, 2023.

(30) Foreign Application Priority Data
Sep. 12, 2022 (JP) 2022-144641

(51) Int. Cl. G01N 15/06; G01K 13/02

(52) U.S. Cl. CPC G01N 15/06; G01K 13/026; G01N 2015/0687

(57) ABSTRACT

A pH of a fluid is estimated without using a pH meter. A pH estimation system for a silica-supersaturated fluid includes: a first flow path that branches off from a pipe in which a fluid containing silica flows, and extracts a part of the fluid; a first measurement unit that is connected to the first flow path via an on-off valve; a retention portion that is connected to the first flow path; a second flow path that is connected to the retention portion and returns the fluid to the pipe; a second measurement unit that is connected to the second flow path via an on-off valve; and a computing device that is electrically connected to the first measurement unit and the second measurement unit, wherein the first measurement unit and the second measurement unit include a fluid temperature measurement device and a silica concentration measurement device; and the computing device stores a relational expression of a rate of decrease in a silica concentration, a silica saturation concentration, a temperature and a pH, and calculates the pH of the fluid, on the basis of measurement results in the first measurement unit and the second measurement unit and the relational expression.

### TECHNICAL FIELD

The present invention relates to a pH estimation device and a pH estimation method for a silica-supersaturated fluid.

### BACKGROUND ART

Conventionally, scale deposition has been a problem in systems including fluid flow systems, such as power generation plants, ship systems, boiler systems, and steel plants. In particular, in a system that uses a geothermal fluid as in a geothermal power generation plant or the like, the deposition of silica-based scale becomes a problem. In order to suppress the deposition of silica scale, in various plants, attempts have been made to measure silica concentration in fluid, and to control the formation and adhesion of the scale on the basis of this measurement result.

An automatic component analysis apparatus for a geothermal steam well is known, which measures a silica content or an electrical conductivity of sample water that is obtained from a two-phase fluid which has been drawn from a steam well, and has a bubbling tank by air or nitrogen gas in a forward stage (for example, see Patent Literature 1). In Patent Literature 1, it is disclosed that the silica concentration and the electrical conductivity can be measured by an automatic analysis of components in steam at an outlet of a steam turbine.

For the purpose of suppressing a disturbance caused by the adhesion of scale, a method is known which includes adding a coloring reagent of which the absorbance in an ultraviolet-visible region varies due to pH variation, to test water which has been collected from boiler water, measuring the absorbance, and determining a pH of the test water on the basis of the absorbance (see, for example, Patent Literature 2). In Patent Literature 2, it is disclosed that a pH of highly alkaline boiler water can be measured without using a pH meter with a glass electrode.

### REFERENCE DOCUMENT LIST

### Patent Documents

Patent Document 1: JP 2009-250760A
Patent Document 2: JP 2021-67430A

### SUMMARY OF THE INVENTION

### Problem to be Solved by the Invention

In the technology disclosed in Patent Literature 1, in a geothermal power generation apparatus, steam is sampled at an outlet of a steam turbine and a silica content is automatically measured. However, silica adhering to the steam turbine before reaching the outlet is excluded from a measurement target; and the silica content is underestimated, and it cannot be said to be accurately measured. It has become clear that various factors relate to the formation of silica scale. Silica concentration in a fluid is an important factor in formation of scale, but the pH of a fluid containing silica is also an important factor.

A pH meter which is commonly used is composed of a porous glass electrode. The heat-resistance temperature of the pH meter is as low as about 80° C. or even less. When an alkaline fluid under conditions in which the pH is 8 or higher is measured by the pH meter, the electrode may dissolve.

In addition, another problem is that when a high-temperature silica-supersaturated fluid is measured with a commercially available pH meter, scale adheres to the porous glass electrode, and thereby, an accurate response cannot be expected. When a pH meter is used for automatic measurement of the pH of a silica-supersaturated fluid, a dedicated system for preventing the formation of the scale is required to increase the measurement accuracy of the pH meter, which leads to a problem of incurring additional cost.

In Patent Literature 2, a technology is disclosed which includes mixing a pH indicator reagent with sample water and measuring the pH on the basis of the absorbance thereof, but is a relatively complicated method. In addition, geothermal water, in particular, is composed not of a single component, but of multiple components, and accordingly, there are possibilities that the color development by a pH indicator reagent will be hindered, and the pH will not be accurately measured. Furthermore, there are possibilities that scale will adhere to a transparent quartz cell for measuring absorbance, and the pH cannot be accurately measured.

The present inventors experimentally and theoretically studied the adhesion of scale, which is caused by polymerization of the silica in the silica-supersaturated fluid, and established a silica deposition prediction equation accompanying a polymerization reaction of silica over time. The present inventors have thereby discovered a system and a method for accurately measuring the pH of the silica-supersaturated fluid, on the basis of actually measured values of the silica concentration and the temperature in the silica-supersaturated fluid, and the relationship among a rate of decrease in a silica concentration, a silica saturation concentration, a temperature and a pH, of the fluid, which has been obtained in advance, and have thereby completed the present invention.

According to one embodiment, the present invention relates to a pH estimation system for a silica-supersaturated fluid, including:
a first flow path that branches off from a pipe through which a fluid containing silica flows, and extracts a part of the fluid;
a first measurement unit that is connected to the first flow path via an on-off valve;
a retention portion that is connected to the first flow path;
a second flow path that is connected to the retention portion and returns the fluid to the pipe;
a second measurement unit that is connected to the second flow path via an on-off valve; and
a computing device that is electrically connected to the first measurement unit and the second measurement unit, wherein
each of the first measurement unit and the second measurement unit includes a fluid temperature measurement device and a silica concentration measurement device; and
the computing device includes a unit that stores a relational expression of a rate of decrease in a silica concentration, a silica saturation concentration, a temperature, and a pH, of a fluid, and that calculates the pH of the fluid, on the basis of measurement results in the first measurement unit and the second measurement unit and the relational expression.

It is preferable that the pH estimation system include a temperature keeping device for the first flow path, the retention portion, and the second flow path.

It is preferable that the pH estimation system include a cooler in a stage preceding the first flow path.

According to another embodiment, the present invention relates to a geothermal power generation facility including a pipe through which a geothermal fluid flows, and a power generation apparatus that generates electric power by rotation of a steam turbine by steam contained in the geothermal fluid, and including the pH estimation system according to any one of the previous descriptions, which is attached to the pipe through which the geothermal fluid flows, at an inlet of the steam turbine.

According to another aspect, the present invention relates to a pH estimation method for a silica-supersaturated fluid flowing through a flow path, including:
a step of measuring a first silica concentration and a first temperature of the silica-supersaturated fluid, at a first measurement point in the flow path;
a step of measuring a second silica concentration and a second temperature of the silica-supersaturated fluid that has reached a second measurement point located downstream of the first measurement point in the flow path;
a step of obtaining a rate of decrease in the silica concentration of the fluid, from a time difference of measurement between the first measurement point and the second measurement point, the first silica concentration, and the second silica concentration; and
a step of obtaining a pH of the silica-supersaturated fluid on the basis of a relationship among the rate of decrease in the silica concentration, the silica saturation concentration, the temperature and the pH, of the fluid, which has been obtained in advance.

In the pH estimation method, it is preferable that the step of obtaining the pH uses the silica deposition prediction equation:

$$dC / dt = - {k ( {C}_{t} - {C}_{e} )}^{n}$$

wherein k is a reaction rate constant of the silica polymerization reaction, Ct is a silica concentration of the fluid at time t, Ce is a silica saturation concentration, and n is an integer of 1 to 5.

In the pH estimation method, it is preferable that a time difference of measurement between the first measurement point and the second measurement point be 5 minutes or longer.

### Effects of the Invention

According to the pH estimation system and the pH estimation method according to the present invention, the pH of the fluid can be estimated on the basis of actually measured values of the silica concentration and the temperature of the fluid, without using a pH meter. Thereby, the pH of the fluid can be accurately obtained, in facilities such as a plant in which the silica-supersaturated fluid flows. Thereby, information useful for the suppression of the silica scale can be obtained by a simple and economically advantageous method.

### BRIEF DESCRIPTION OF THE DRAWINGS

FIG. 1 shows a view conceptually showing a pH estimation system according to a first embodiment of the present invention.

FIG. 2 shows a schematic graph showing a silica deposition prediction equation.

FIG. 3 shows a graph showing a relationship between a temperature and a dissolved silica concentration in acidic, neutral, and alkaline pHs.

### MODE FOR CARRYING OUT THE INVENTION

Embodiments of the present invention will be described below with reference to the drawings. However, the present invention is not limited to the embodiments described below.

### First Embodiment: pH Estimation System and Method

The present invention, according to one embodiment, relates to a pH estimation system and method. The pH estimation system is a system for estimating a pH of a silica-supersaturated fluid, and includes:
a first flow path that branches off from a pipe through which a fluid containing silica flows, and extracts a part of the fluid;
a first measurement unit that is connected to the first flow path via an on-off valve;
a retention portion that is connected to the first flow path;
a second flow path that is connected to the retention portion and returns the fluid to the pipe;
a second measurement unit that is connected to the second flow path via an on-off valve; and
a computing device that is electrically connected to the first measurement unit and the second measurement unit.

The pH estimation system and method according to the present embodiment relate to a system and method for estimating the pH of the fluid from the fluid temperature and the silica concentration at two or more measurement points in the fluid, on the basis of the calculation equation and accumulated data, without directly measuring a pH of the fluid that is a measurement target sample.

In the present embodiment, a fluid to be measured is the silica-supersaturated fluid. In more detail, the fluid refers to a fluid that contains silicic acid in excess of its solubility at the fluid temperature, and contains chemical species containing Si and OH, which include Si(OH)₄, Si(OH)₃O⁻, SiO₂(OH)₂²⁻, Si₂O₂(OH)₅, and/or Si₂O₃(OH)₄²⁻, but these are not limited thereto. In addition, the term “fluid” means that the fluid may be a liquid such as water, or may be an object forming a two-phase flow which is a mixture of a liquid such as water and a gas such as water vapor.

The silica-supersaturated fluid to be measured may also be a fluid that flows through a plant. The plant is a plant through which a fluid containing silica flows, and which can cause a shutdown or a malfunction due to the adhesion of silica scale. Examples of the plant include a geothermal power plant, a boiler system, a system including a cooling water pipe, and a water treatment system, but this is not limited to these plants and systems. In addition, the fluid flowing through the plant does not need to be the silica-supersaturated fluid. When the fluid flowing through the plant is a fluid that does not reach oversaturation, the fluid can be an object to be measured after having been brought into an supersaturated state by an operation of lowering a temperature of the fluid by a cooler that is optionally provided in the pH estimation system according to the present embodiment. Details will be described later.

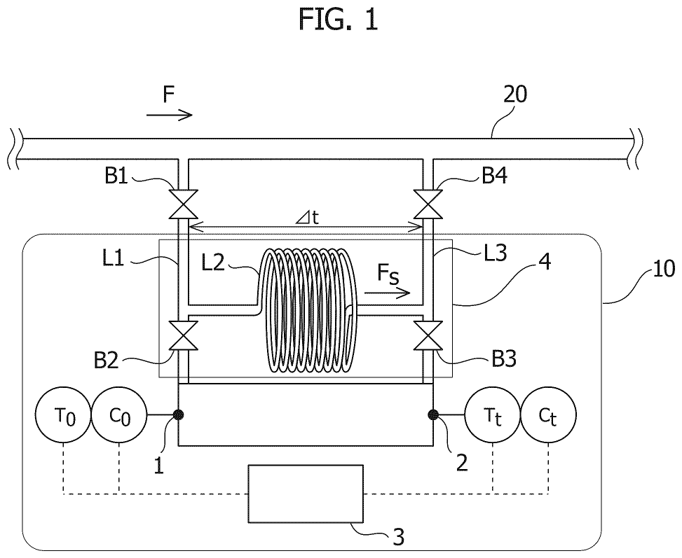

The present invention will be described below with reference to an example of a geothermal fluid which flows in a geothermal power generation facility, but the present invention is not limited to the estimation of the pH of the geothermal fluid. FIG. 1 shows a diagram conceptually showing a pH estimation system according to a first embodiment. A pH estimation system 10 shown in FIG. 1 mainly includes a first flow path L1, a retention portion L2, a second flow path L3, a first measurement unit 1, a second measurement unit 2, and a computing device 3.

According to the present embodiment, the pH estimation system 10 can be configured by a flow path branched from a pipe 20 through which the silica-supersaturated fluid flows, and a measurement unit for physical properties of the fluid flowing through the flow path. Hereinafter, the silica-supersaturated fluid will be referred to as “fluid” in an abbreviated form. The pipe 20 may be any pipe and is not particularly limited. In the geothermal power generation facility, the pipe may be a pipe at a place in which it is advantageous to obtain a pH of the fluid, and may be a pipe particularly in a stage preceding the steam turbine.

The first flow path L1 is a pipe that branches off from pipe 20, and introduces a part of the fluid flowing through the pipe 20 into the pH estimation system 10. The first flow path L1 may be any path so long as the flow path can send a part of the fluid flowing through the pipe 20 to the downstream first measurement unit 1 in a state of maintaining the temperature, and may be, for example, a pipe made from a metal such as a carbon steel or a stainless steel.

A diameter of first flow path L1 is not particularly limited, and for example, an outer diameter can be 2 to 12.7 mm, and may be the same diameter as the retention portion L2, which will be described later. An inlet valve B1 can be provided at an inlet of the first flow path L1, in other words, between the pipe 20 and the first flow path L1. Thereby, the pH estimation system 10 can be configured so that only when the valve B1 is open, a part of the fluid flowing through the pipe 20 flows into the first flow path L1. A length of the first flow path L1 is defined as a length from the inlet valve B1 to a valve B2 of the inlet of the first measurement unit 1. The length of the first flow path L1 is not particularly limited, but it is preferable that the length of the flow path be as short as possible, in order to suppress heat radiation from the flow path and minimize a temperature drop. For example, the length can be set at 0.1 to 1 m.

The first measurement unit 1 is a portion which actually measures the temperature and silica concentration of the fluid at the inlet of the pH estimation system 10. The first measurement unit 1 is connected to the first flow path L1 via the valve B2. In addition, when the valve B2 is open, the pH estimation system 10 can send a part of the fluid which has been extracted from the pipe 20 to the first measurement unit 1, and can actually measure the temperature and the silica concentration of the fluid. Before the measurement is performed, the valves B1 and B4 are opened to allow the fluid to flow, and after the flow has been stabilized, B2 is opened, and the fluid is sampled. After sampling of a predetermined amount of fluid has been completed, B2 is closed. The amount of fluid required for measurement in the first measurement unit 1 depends on the configuration of the first measurement unit 1, but can be set to 5 to 10 mL.

The first measurement unit 1 is provided with a thermometer for the purpose of measuring a first temperature T₀ of the fluid. It is preferable to measure the temperature of the fluid so that the thermometer does not contact with the fluid, and for example, it is preferable to measure the temperature of a sampled fluid from the outside of a container. The reason is to eliminate influence of scale formation on the thermometer. Alternatively, it is acceptable to insert the thermometer into the sampled fluid and directly measure the temperature of the fluid, but in this case, it is preferable that the thermometer be provided with such a structure that a portion which comes into contact with the fluid can be cleaned to prevent the adhesion of scale. In addition, the first measurement unit 1 is provided with an online silica concentration measurement device for the purpose of measuring a first silica concentration Co of the fluid. As for a structure of the online silica concentration measurement device, an automatic device can be used which measures the silica concentration with the use of a molybdenum yellow method. As an automatic measurement device for the silica concentration, for example, a Mettler-Toledo device can be used, but the device is not limited to a specific device.

It is desirable to provide a waste tank that can store a fluid which has been used for the actual measurement of the temperature and the silica concentration in the first measurement unit 1, in the on-line silica concentration measurement device, collect the waste water at the time of maintenance of the plant or the device, according to regulations, and perform final disposal. The first measurement unit 1 can be regarded as a first measurement point for obtaining the first silica concentration Co and the first temperature T₀, in the pH estimation method, which will be described later.

The retention portion L2 that branches off from the first flow path L1 is a relatively long flow path existing between the first flow path L1 and the second flow path L3. The retention portion L2 is provided for the purpose of increasing a time interval Δt until a fluid Fs extracted from the pipe 20 reaches the second measurement unit 2 from the first measurement unit 1, preferably, to 5 minutes or longer. The type and the diameter of the pipe constituting the retention portion L2 may be the same as those of the pipe constituting the first flow path L1, and is preferably formed from a material to which the scale is less likely to adhere and which has heat resistance. A structure of the retention portion L2 is not particularly limited so long as the structure can secure the above time interval Δt, and can be, for example, a spiral structure (also referred to as a loop structure). Due to the retention portion L2 which is formed into a spiral structure, the flow path length can be secured, and the pH estimation system 10 can be structured compact. A length of the retention portion L2 may be any length so long as the portion can secure the above time interval Δt, and is not particularly limited, but can be set at about 3 to 300 m, for example.

The second flow path L3 is a flow path for returning the fluid Fs flowing through the first flow path L1 and the retention portion L2, to the pipe 20 through which the silica-supersaturated fluid flows. The preferable type, diameter and length of the pipe constituting the flow path may be the same as those of the first flow path L1. In addition, the second flow path L3 is connected to the pipe 20 through which the silica-supersaturated fluid flows, and a valve B4 is provided at an outlet of the second flow path L3. It is preferable to set a connection position between the second flow path L3 and the pipe 20, on a downstream side of the flow of a fluid F which flows through the pipe 20, with respect to the connection position between the first flow path L1 and the pipe 20.

The second measurement unit 2 actually measures a temperature and silica concentration of the fluid Fs that has flowed through the retention portion L2 and has flowed into the second flow path L3, in other words, the temperature and silica concentration of the fluid Fs at the outlet of the pH estimation system 10. The second measurement unit 2 is connected to second flow path L3 via a valve B3. In addition, when the valve B3 is open, the pH estimation system 10 sends a part of the fluid which has flowed through the retention portion L2, to the second measurement unit 2, and can actually measure the temperature and silica concentration of the fluid. An operation at the time of sampling may be the same as in the first measurement unit. Accordingly, before the measurement is performed, the valves B1 and B4 are opened to allow the fluid to flow, and after the flow has been stabilized, B3 is opened, and the fluid is sampled. After sampling of a predetermined amount of the fluid has been completed, B3 is closed. The amount of the fluid required for the measurement in the second measurement unit 2 can also be similar to that in the first measurement unit 1.

The computing device 3 stores a relationship among the rate of decrease in the silica concentration, the silica saturation concentration, the temperature, and the pH, of the fluid, which has been obtained in advance, and calculates a pH of the fluid, on the basis of the measurement results in the first measurement unit and the second measurement unit. The computing device 3 may be, for example, a computer, but it is not limited to a specific device. The computing device 3 is electrically connected to the first measurement unit 1 and the second measurement unit 2, in a form capable of receiving data of the silica concentration and the temperature, from the first measurement unit 1 and the second measurement unit 2. The computing device 3 can also optionally function as a display device that displays an estimation result of the pH, and may be configured to be capable of monitoring the estimation result of the pH.

A temperature maintaining device 4 covers at least a periphery of the first flow path L1, the retention portion L2 and the second flow path L3, and prevents a temperature of the fluid passing through the flow paths from decreasing. The temperature maintaining device 4 may be a heat insulating material provided around each of the flow paths, or may be a device such as a thermostatic bath that heats or cools the flow path from the outside and maintains the fluid at a specific temperature.

According to one embodiment, the pH estimation system 10 may include a cooler at the inlet of the first flow path L1 (not illustrated). The cooler may be any cooler so long as the cooler can reduce a temperature of the fluid which flows through the first flow path L1. When the fluid flowing through the pipe 20 is not in a silica-supersaturated state, it is possible to lower the temperature of the fluid, thereby generating a pseudo silica-supersaturated fluid, and obtain a state in which the pH can be estimated. When the pH estimation system 10 includes the cooler, the pH estimation system 10 can also be configured to be capable of controlling a temperature after cooling, on the basis of the temperature measurement result in the first measurement unit 1, and may include a control device.

According to another embodiment, the pH estimation system 10 can also be installed at the pipe 20 through which the silica-supersaturated fluid flows so as to be removable. In this case, it is possible to provide a port which communicates with the first flow path and a port which communicates with the second flow path, to the pipe 20 through which the silica-supersaturated fluid flows. Accordingly, it becomes possible to measure the pH in any pipe provided with such a port, in the geothermal power generation facility.

According to yet another embodiment, the pH estimation system 10 may further include an additional measurement unit and an additional retention portion. For example, the pH estimation system 10 may include, in place of the retention portion L2 shown in the figure, a first retention portion which is connected to the first flow path L1; a second retention portion which is located downstream of first retention portion, and is connected to the second flow path L3; and a third measurement unit which is connected so as to be capable of measuring a temperature and silica concentration of a fluid between the first retention portion and the second retention portion. As for similar configurations, the pH estimation system 10 may include three retention portions and two additional measurement units, or may include more retention portions and additional measurement units. An advantage of having a plurality of measurement units is that it becomes possible to enhance the accuracy of the pH estimation method which will be described later.

Next, the invention according to the first embodiment will be described from the viewpoint of the pH estimation method. The pH estimation method according to the present embodiment is a pH estimation method for a silica-supersaturated fluid flowing through a flow path, and includes the following steps:
(1) a step of measuring a first silica concentration and first temperature of the silica-supersaturated fluid, at a first measurement point in the flow path;
(2) a step of measuring a second silica concentration and second temperature of the silica-supersaturated fluid that has reached a second measurement point located downstream of the first measurement point in the flow path;
(3) a step of obtaining a rate of decrease in the silica concentration from a time difference of measurement between the first measurement point and the second measurement point, the first silica concentration, and the second silica concentration; and
(4) a step of obtaining the pH of the silica-supersaturated fluid on the basis of the relationship among the rate of decrease in the silica concentration, a silica saturation concentration, the temperature and the pH, which has been obtained in advance.

The pH estimation method according to the present embodiment is based on an experimental fact and a theoretical basis that, in a fluid in which silica is supersaturated, a polymerization reaction of silica progresses with the passage of time, the silica concentration in the fluid decreases, and the rate of decrease depends on the temperature and the pH. When the temperature can be maintained constant and the change in silica concentration with time and the temperature can be actually measured, the pH can be estimated on the basis of the silica deposition prediction equation which the present inventors have experimentally established.

The pH estimation method according to the present embodiment uses the following silica deposition prediction equation (equation (I)) and equation of the reaction rate constant k (equation (II)):

$$dC / dt = - {k ( {C}_{t} - {C}_{e} )}^{n} \qquad ( I )$$

wherein k is the reaction rate constant of a silica polymerization reaction, Ct is a silica concentration of the fluid at time t, Ce is the silica saturation concentration, and n is an integer of 1 to 5; and

$$k = A \cdot exp ( - {E}_{a} / RT ) \qquad ( II )$$

wherein A is a frequency factor, Ea is an activation energy, R is a gas constant, and Tis a temperature of the silica polymerization reaction, which is a temperature of a fluid exemplified by geothermal water.

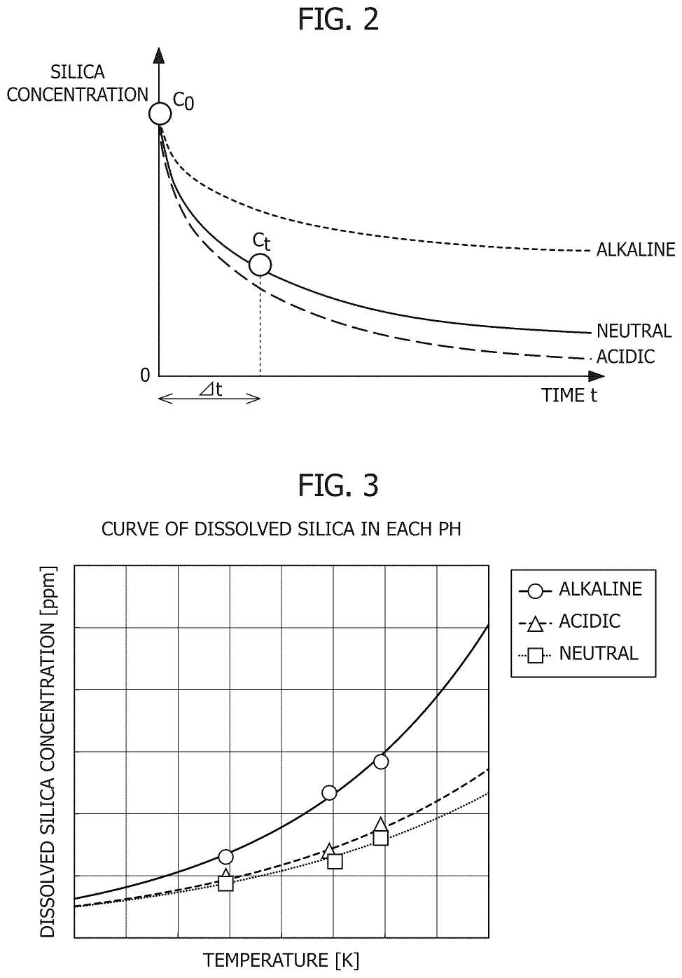

FIG. 2 shows a graph schematically showing the silica deposition prediction equation (equation (I)) in the case in which the temperature T is constant, and is a graph in the case of n=2. The prediction curves in acidic, neutral, and alkaline regions are curves which have been experimentally derived, and the value of k varies depending on the pH. By actual measurement of the silica concentration Co at time t=0 and the silica concentration Ct at time t after Δt, in the silica-supersaturated fluid, the rate of decrease in the silica concentration is obtained from FIG. 2, which is expressed by dC/dt; and the rate of decrease varies depending on the pH. Referring to a definition equation of the reaction rate constant k (equation (II)), the frequency factor A and the activation energy Ea are values depending on the pH, and accordingly, it can also be theoretically understood that the reaction rate constant k is a term depending on the pH, in the equation (I). The frequency factor A can be calculated by fitting, and Ea can be theoretically calculated from an elementary reaction. The elementary reaction refers to each of reactions in a process through which the scale of silica grows in the silica polymerization reaction. For example, a reaction through which silica monomers grow to a silica tetramer includes a combination of the following reactions. Monomer+monomer→dimer, monomer+dimer→trimer, monomer+trimer→tetramer, and dimer+dimer→tetramer. Each of the reactions is an elementary reaction. The silica of a monomer is defined to have a structure having one Si atom, and the silica of a dimer is defined to have a structure having two Si atoms.

In FIG. 2, the silica deposition prediction curves are schematically shown for representative three pHs in acidic, neutral, and alkaline regions, but in practice, it is preferable to experimentally obtain a silica deposition prediction curve in advance, in a pH range of 5.5 to 9, for example, in increments of 0.1. Similarly, it is preferable to experimentally obtain the silica deposition prediction curve in advance, at a temperature in a range of 25° C. to 200° C., for example, in increments of 15° C.

In the equation (I), Ce is a silica saturation concentration, and the silica saturation concentration is also a value depending on the pH and the temperature. The temperature dependency of the silica saturation concentration can also be experimentally obtained in advance. FIG. 3 shows a graph showing a relationship between the temperature and dissolved silica concentration (saturation concentration) of the fluid in acidic, neutral, and alkaline regions. The plots in the graph are examples of experimental data, and they represent the prediction curve concerning the temperature dependency of the silica saturation concentration. It is preferable to experimentally also obtain the prediction curve of the silica saturation concentration in advance, for example, in a pH range of 5.5 to 9, and for example, in increments of 0.1.

Each of the steps will be described below with the use of the pH estimation system shown in FIG. 1. However, the above steps are not necessarily limited to a method which is performed using the pH estimation system shown in FIG. 1.

In the step (1), a first silica concentration Co and a first temperature T₀ of the silica-supersaturated fluid are measured at a first measurement point in the flow path. The first silica concentration and the first temperature can be measured by the first measurement unit 1 shown in FIG. 1. The obtained actually measured values can be transmitted electrically to the computing device 3.

There is a case in which a step of obtaining a silica-supersaturated fluid is performed as a preparation step, before the step (1) is performed. For example, when the fluid containing silica is unsaturated, it is preferable to lower the temperature of the fluid, thereby converting the fluid into a pseudo supersaturated state, and then perform the measurement.

In the step (2), a second silica concentration Ct and a second temperature Tt of the silica-supersaturated fluid that has reached the second measurement point located downstream of the first measurement point in the flow path are measured. The time required for the fluid to reach the second measurement point from the first measurement point can also be regarded as a time difference of measurement between the first measurement point and the second measurement point, and is represented by Δt. The Δt can vary depending also on a shape of and pressure in the flow path, and accordingly, it is preferable to obtain the Δt in advance by a preliminary experiment or calculation. It is preferable to determine the first measurement point and the second measurement point so that the Δt becomes 5 minutes or longer, and when the apparatus shown in FIG. 1 is used, it is preferable to design a length of the flow path of the retention portion L2 so that the Δt becomes 5 minutes or longer. The reason why the Δt is set in this way is because in the pH estimation method in the present embodiment, the rate of decrease in the silica concentration is used as one of the parameters, and the estimation is performed on the basis of the rate; and accordingly, the silica polymerization reaction rapidly changes, and the difference among the parameters tends to be easily grasped. The method for measuring the second silica concentration Ct and the second temperature Tt can be the same as in the step (1), and can be performed by the second measurement unit 2 shown in FIG. 1. The obtained actually measured values can be transmitted electrically to the computing device 3.

In carrying out the method of the present invention, the temperature of the fluid to be measured for the silica concentration and temperature is held constant. In more detail, the temperature of the fluid is maintained constant while the fluid passes through the first measurement point and reaches the second measurement point. When the apparatus shown in FIG. 1 is used, the temperature of the first flow path L1, the retention portion L2, and the second flow path L3 can be maintained constant by the temperature keeping device 4. In the present steps (1) and (2), the first temperature T₀ and the second temperature Tt are measured, but this measurement is confirmatory, and it is preferable to control the temperature of the fluid so as to become T₀=Tₜ.

In the step (3), the rate of decrease in the silica concentration is obtained from a time difference of measurement between the first measurement point and the second measurement point, the first silica concentration, and the second silica concentration. The rate of decrease in silica concentration is represented by dC/dt, and the rate of decrease in the silica concentration can be obtained by calculation with the use of the Co obtained in the step (1), the Ct obtained in the step (2), and the Δt.

In the step (4), the pH is calculated on the basis of the relationship among the rate of decrease in the silica concentration, a silica saturation concentration, the temperature and the pH, which has been obtained in advance. To be more specific, the step (4) includes: a step (a) of inputting the calculation result of the step (3) into the silica deposition prediction equation; a step (b) of calculating values of a reaction rate constant k and a silica saturation concentration Ce that fit to the silica deposition prediction equation; and a step (c) of extracting a pH that gives the calculated reaction rate constant k and the silica saturation concentration Ce.

In the step (a), the value of dC/dt obtained in the step (3) and the second silica concentration Ct obtained in the step (2) are input to the equation (I). The equation (I) can be calculated with the use of any integer of 1 to 5 as n, and it is preferable to set n=2.

In the step (b), the calculation is performed by varying the values of k and Ce for the equation (I) into which the dC/dt and the Ct have been input in the step (a), and the values of k and Ce satisfying the equation (I) are calculated. The temperature T in the reaction rate constant k is calculated with the use of the actually measured first temperature T₀ (where the same applies to the second temperature Tₜ). Once the temperature is determined, k and Ce can be determined for each of the pHs, on the basis of experiments.

In the step (c), for the equation (I) into which the dC/dt and the Ct have been input in the step (a), a pH is extracted that gives the reaction rate constant k and the silica saturation concentration Ce calculated in the step (b). As the data of the extraction source, the prediction curve of the pH and temperature dependency of the silica saturation concentration can be used, which has been obtained in advance as illustrated in FIG. 3.

In addition, in the estimation method according to the present embodiment, the steps (3) and (4) can be regarded as a pH estimation program, and the present invention includes the following.

A pH estimation program for a silica-supersaturated fluid flowing through a flow path and for causing a computer to perform the following steps that include:
a step of acquiring a first silica concentration and a first temperature of the silica-supersaturated fluid, at a first measurement point in the flow path through which the silica-supersaturated fluid flows;
a step of acquiring a second silica concentration and a second temperature of the silica-supersaturated fluid that has reached a second measurement point located downstream of the first measurement point in the flow path;
a step of obtaining a rate of decrease in the silica concentration from a time difference of measurement between the first measurement point and the second measurement point, the first silica concentration, and the second silica concentration; and
a step of obtaining a pH of the silica-supersaturated fluid on the basis of the relationship among the rate of decrease in the silica concentration, the silica saturation concentration, the temperature, and the pH, which has been obtained in advance.

An operation of each of the steps is as described in the pH estimation method.

According to the pH estimation method of the present embodiment, the pH of the silica-supersaturated fluid can be obtained by measurements of the silica concentration and the temperature of the silica-supersaturated fluid, at two positions.

### Second Embodiment: Geothermal Power Generation Facility

According to a second embodiment, the present invention relates to a geothermal power generation facility. The geothermal power generation facility includes a pipe through which a geothermal fluid flows, and a power generation apparatus that generates electric power by rotation of a steam turbine by steam contained in the geothermal fluid, and includes the pH estimation system according to the first embodiment, which is attached to the pipe through which the geothermal fluid flows, at an inlet of the steam turbine.

The geothermal power generation facility is a facility that generates electric power while using the geothermal fluid as a power source, and mainly includes: a production well; a steam separator; a steam turbine; a reduction well; and pipes that connect these components and through which the geothermal fluid flows. In the geothermal power generation facility according to the second embodiment, the pH estimation system according to the first embodiment is connected to the pipe. Preferably, the pH estimation system according to the first embodiment is connected to a pipe that is arranged in a stage preceding the steam turbine, and supplies the geothermal fluid to the steam turbine.

The flow of the geothermal fluid in a geothermal power generation system will be described. The production well is a well that spouts the geothermal fluid which is hot water, steam, or a mixture thereof, and exists in a geothermal reservoir in the ground to above-ground. The geothermal fluid spouted from the production well is separated into steam as a gas component and hot water as a liquid component, by the steam separator. The separated steam is led to the steam turbine and is used to rotate the turbine, and thereby produces electricity in an electric power generator. The steam that has worked in the turbine is cooled and condensed, and is sent to a cooling tower. On the other hand, the hot water separated by the steam separator is returned to the reduction well, via a heat exchanger, a hot water pit and the like.

In the case of a binary power generation system, a second steam separator is provided at a stage subsequent to the steam separator, and the steam separated by the second steam separator heats a medium having a low boiling point. The heated medium having the low boiling point is used for the rotation of a second turbine. In some cases, the medium having the low boiling point is used while being circulated and being repeatedly evaporated and condensed, and the hot water separated by the steam separator is used for heating the medium having the low boiling point. The geothermal power generation facility according to the present embodiment describes a configuration common to the binary power generation system and the system that operates a turbine only by the geothermal steam; and both of the binary power generation system and the system that operates the turbine only by the geothermal steam fall within the scope of the present invention.

The geothermal power generation facility according to the second embodiment of the present invention includes the pH estimation system illustrated in FIG. 1. When information about the pH of the geothermal fluid is necessary, the valves B1 and B4 can be opened, and the pH can be estimated. Thereby, it becomes possible to accurately grasp the pH of the geothermal fluid in the geothermal power generation facility, and perform an operation necessary for suppressing the adhesion of silica scale.

### REFERENCE SYMBOL LIST

L1 first flow path
L2 retention portion
L3 second flow path
B1, B2, B3 and B4 valve
1 first measurement unit
2 second measurement unit
3 computing device
4 temperature keeping device
10 pH estimation system
20 pipe
F fluid
Fs fluid extracted from pipe

1. A pH estimation system for a silica-supersaturated fluid, comprising:
a first flow path that branches off from a pipe through which a fluid containing silica flows, and extracts a part of the fluid;
a first measurement unit that is connected to the first flow path via an on-off valve;
a retention portion that is connected to the first flow path;
a second flow path that is connected to the retention portion and returns the fluid to the pipe;
a second measurement unit that is connected to the second flow path via an on-off valve; and
a computing device that is electrically connected to the first measurement unit and the second measurement unit, wherein
each of the first measurement unit and the second measurement unit comprises a fluid temperature measurement device and a silica concentration measurement device; and
the computing device is configured to store a relational expression of a rate of decrease in a silica concentration, a silica saturation concentration, a temperature, and a pH, of a fluid, and to calculate the pH of the fluid, on the basis of measurement results in the first measurement unit and the second measurement unit and the relational expression.

2. The pH estimation system according to claim 1, comprising a temperature maintaining device for the first flow path, the retention portion, and the second flow path.

3. The pH estimation system according to claim 1, comprising a cooler in a stage preceding the first flow path.

4. A geothermal power generation facility, comprising
a pipe through which a geothermal fluid flows, and
a power generation apparatus that generates electric power by rotation of a steam turbine by steam contained in the geothermal fluid, and comprising
the pH estimation system according to claim 1, which is attached to the pipe through which the geothermal fluid flows, at an inlet of the steam turbine.

5. A pH estimation method for a silica-supersaturated fluid flowing through a flow path, comprising:
a step of measuring a first silica concentration and a first temperature of the silica-supersaturated fluid, at a first measurement point in the flow path;
a step of measuring a second silica concentration and a second temperature of the silica-supersaturated fluid that has reached a second measurement point located downstream of the first measurement point in the flow path;
a step of obtaining a rate of decrease in a silica concentration from a time difference of measurement between the first measurement point and the second measurement point, the first silica concentration, and the second silica concentration; and
a step of obtaining a pH of the silica-supersaturated fluid on the basis of a relationship among the rate of decrease in the silica concentration, a silica saturation concentration, the temperature, and the pH, which has been obtained in advance.

6. The pH estimation method according to claim 5, wherein
the step of obtaining the pH uses the silica deposition prediction equation:
$$dC / dt = - {k ( {C}_{t} - {C}_{e} )}^{n}$$
wherein k is a reaction rate constant of a silica polymerization reaction, Cₜ is a silica concentration of the fluid at time t, C_{c} is a silica saturation concentration, and n is an integer of 1 to 5.

7. The pH estimation method according to claim 5, wherein the time difference of measurement between the first measurement point and the second measurement point is 5 minutes or longer.
